(19) United States
(12) Patent Application Publication
(10) Pub. No.: US 2017/0362558 A1
(43) Pub. Date: Dec. 21, 2017

(54) BIOLOGICAL SAMPLE ACTUATOR

(71) Applicant: OXFORD UNIVERSITY INNOVATION LIMITED, Oxford (GB)

(72) Inventors: Jonathan Joshua Burton, Kington (GB); Malcolm McCulloch, Oxford (GB); Mark Stuart Thompson, Oxford (GB); Chris Stevens, Oxford (GB)

(21) Appl. No.: 15/535,012

(22) PCT Filed: Dec. 7, 2015
(86) PCT No.: PCT/GB2015/053738

§ 371 Date: Jun. 9, 2017

(30) Foreign Application Priority Data
Dec. 11, 2014 (GB) 1422016.4

(51) Int. Cl. C12M 1/42; G01N 3/38

(52) U.S. Cl. CPC C12M 35/04; G01N 3/38

(57) ABSTRACT

An apparatus is provided for mechanically loading a biological sample. The apparatus comprises: a container for housing the biological sample; a ferromagnetic element, for attachment to the biological sample within the container; and a solenoid for generating a magnetic field within the container, so as to apply a force to the ferromagnetic element. The solenoid is configured, when energised by a constant current, to produce a force on the ferromagnetic element that varies by less than a predetermined amount over a predetermined range of movement of the magnet within the container. A method of mechanically loading a biological sample is also disclosed.

The invention relates to an actuator for mechanically loading a biological sample.

Biological tissue may respond to mechanical loading. For example, exercise can significantly enhance bone strength at loaded sites in children¹. Collagen turnover in tendons in humans is related to levels of activity: inactivity tending to decrease collagen turnover². ¹ Nikander, Riku, et al. “Targeted exercise against osteoporosis: A systematic review and meta-analysis for optimising bone strength throughout life.” BMC medicine 8.1 (2010): 47² Kjær, Michael. “Role of extracellular matrix in adaptation of tendon and skeletal muscle to mechanical loading.” Physiological reviews 84.2 (2004): 649-698)

In the field of regenerative medicine there is a need to understand the mechanisms by which tissue responds to physical activity. One aspect of this response is how tissue responds to mechanical loading. Understanding the response of tissue in vivo is complicated, because the environment is very complex and difficult to control. The ability to subject tissue to mechanical loading in vitro potentially enables more control over the environment.

One application for in vitro mechanical loading of biological samples is to perform basic science, allowing specific hypotheses to be tested in a controlled manner. Mechanical loading of tissues may provide a more representative arrangement for drug testing on such tissues, contributing to reducing and replacing animal testing in medicine. Furthermore, arrangements for mechanical loading of tissue may facilitate improved tissue engineering.

Preferably, an arrangement for mechanical loading would be compatible with maintaining a sterile environment around the biological sample under test, and allow a controlled temperature to be maintained. Furthermore, it is preferable that relatively high cycles of reversed stress can be applied to the sample. Relatively high frequency tissue loading is an active area of research, with whole body vibration at 30 Hz having shown promise in increasing tendon stiffness and bone mass^{3,4}. ³ Rubin C, Turner A S, Bain S, Mallinckrot C, McLoed K. Low mechanical signals strengthen long bones. Nature 2001; 412:603-4.⁴ Sandhu E, Mile J D, Dauhners L E, Keller B V, Weinhold P S. Whole body vibration

An arrangement for mechanical loading has been reported, in which a tendon is mechanically loaded using a magnet and solenoid⁵. A magnet was secured to the tendon, and then placed near a solenoid. The force applied to the tendon was varied by varying the current through the solenoid. A load cell was used to determine the applied load. ⁵ Adekanmbi, Isaiah, Sarah Franklin, and Mark S. Thompson. “A novel in vitro loading system for high frequency loading of cultured tendon fascicles.” Medical engineering & physics 35.2 (2013): 205-210.

WO2013095834 discloses a system for applying mechanical stimulation to a biological sample. The biological sample is clamped at each end to a holder, and a force is directly exerted on the sample via the holder.

A simplified arrangement would be preferable, as would an arrangement with lower cost. One problem with prior art arrangements for mechanical tissue loading is consistency of loading.

An arrangement for mechanical tissue loading that addresses at least some of the above mentioned problems is desired.

According to the invention, there is provided an apparatus for mechanically loading a biological sample, comprising: a container, for housing the biological sample; a ferromagnetic element, for attachment to the biological sample within the container; and a solenoid for generating a magnetic field within the container, so as to apply a force to the ferromagnetic element, the solenoid having a solenoid axis; wherein the solenoid is configured, when energised by a constant current, to produce a force on the ferromagnetic element that varies by less than a predetermined amount over a predetermined range of movement of the ferromagnetic element within the container.

The container may be hermetically sealable. The container may comprise gas exchange means. The gas exchange means may comprise small holes, or a gas permeable membrane. The gas exchange means may permit oxygenation of cultured specimens.

increases area and stiffness of the flexor carpi ulnaris tendon in the rat. J Biomech 2011; 44:1189-91

The predetermined amount may be 50%, 30%, 20%, 10%, 5% or 2%.

The predetermined range of movement may be at least 50 mm, 40 mm, 30 mm, 20 mm, 10 mm or 5 mm.

The improved uniformity of force over distance results in improved consistency of mechanical stimulation of a biological sample. The impact of any errors in the initial position of the ferromagnetic element within the solenoid are minimised. Similarly, the effect of any changes in dimension of the sample on the mechanical loading of the sample.

The solenoid may be configured to produce a field gradient that varies by less than a further predetermined amount over a further predetermined distance along the solenoid axis.

The further predetermined amount may be 30%, 20%, 10%, 5% or 2%.

The further predetermined distance may be at least 50 mm, 40 mm, 30 mm, 20 mm, 10 mm or 5 mm.

Where the ferromagnetic element is small (relative to the range of movement), one way of approximating a force that is constant over a range of movement is to configure the solenoid to produce a field gradient that is relatively uniform over the desired range of movement.

The solenoid can be configured with the necessary field distribution to achieve the desired uniformity of force (for a given current) over a desired range of movement of the ferromagnetic element in a number of ways.

The solenoid may comprise a winding having a radius that varies with position along the solenoid axis. Varying the radius of the winding may be achieved by varying the radius of a former or bobbin around which the winding of the solenoid is wound.

The radius of the winding may increase with distance from the centre of the solenoid. This is a convenient way of providing for a more uniform field gradient.

The solenoid may comprise a winding that has a number of winding layers that varies with position along the solenoid axis. For instance, the number of winding layers may be greater in the centre of the solenoid, so as to produce a more uniform field gradient within the solenoid.

The solenoid may comprise a winding has a pitch that varies with position along the solenoid axis. For example, the pitch of the winding may decrease away from the centre of the solenoid, so as to produce a more uniform field gradient within the solenoid.

The apparatus may comprise means for cooling the solenoid by forced convection of a cooling fluid. Actively cooling the solenoid enables it to handle greater currents without overheating, increasing the amount of force that can be applied on the ferromagnetic element.

The fluid used for cooling may be air or water, or any other suitable fluid.

The container may be a sterile container.

The container may comprise a removable lid. The removeable lid may comprise the gas exchange means. For instance, the lid may be pierced by one or more holes. The holes may be less than 2 mm, 1 mm, 0.5 mm or 0.25 mm in diameter.

The lid may comprise attachment means, for attachment to the biological sample. The attachment means may comprise a post, clamp, grip or the like.

The ferromagnetic element may comprise a permanent magnet. The use of a magnet may enhance the amount of force that can be applied on the biological sample by the solenoid. A high field strength permanent magnet is particularly suitable for increasing the force.

The ferromagnetic element may comprise a biologically inert coating. The use of a biologically inert coating may facilitate mechanical loading of tissue, without substantially affecting the viability of the tissue.

The coating may be a polymeric material, for instance a flourinated polymer such as polytetraflouroethylene (PTFE).

The solenoid may have a hole along the axis for at least partially receiving the container so that the ferromagnetic element within the container is within the solenoid.

The diameter of the hole may be at least 10 mm. The diameter of the hole may be at least 20 mm, 30 mm, 40 mm, or 50 mm.

The depth of the hole may be at least 20 mm. The hole may be a through hole. The depth of the hole may be at least 30 mm, 40 mm, 50 mm. 75 mm or 100 mm.

The length of the solenoid may be at least 50 mm, 60 mm, 75 mm or 100 mm.

According to second aspect of the invention, there is provided a method of mechanically loading a biological sample, comprising: attaching a first portion of a the biological sample to a fixed object; attaching a ferromagnetic element to a second portion of the biological sample; positioning the ferromagnetic element within a working region of a solenoid; and exerting a force on the ferromagnetic element using a solenoid; wherein the solenoid is configured, when energised by a constant current, to produce a force on the ferromagnetic element that varies by less than a predetermined amount over a predetermined range of movement of the ferromagnetic element within the container.

Any of the features of the first aspect may be included in the second aspect of the invention.

The method may comprise using the apparatus according to any preceding claim.

The method may include a step of testing a substance for use a medicament on the biological sample while it is loaded periodically using the solenoid.

The biological sample may be tissue, and the method may comprise conditioning the tissue using the force exerted by the solenoid.

Example embodiments of the invention will now be described, with reference to the accompanying drawings, in which:

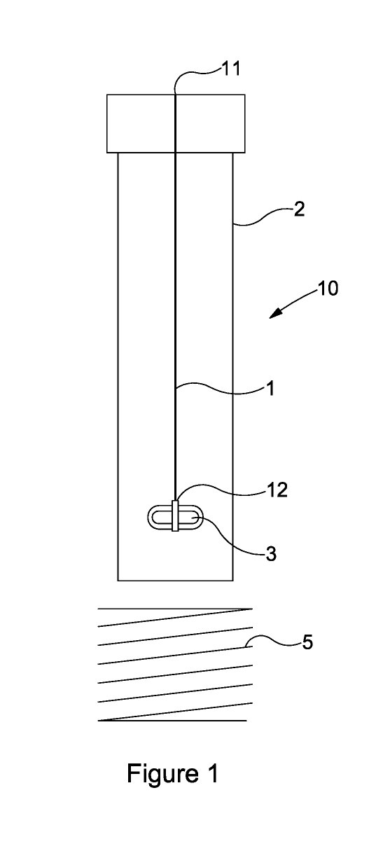

FIG. 1 is a schematic diagram of an apparatus according to an embodiment of the invention;

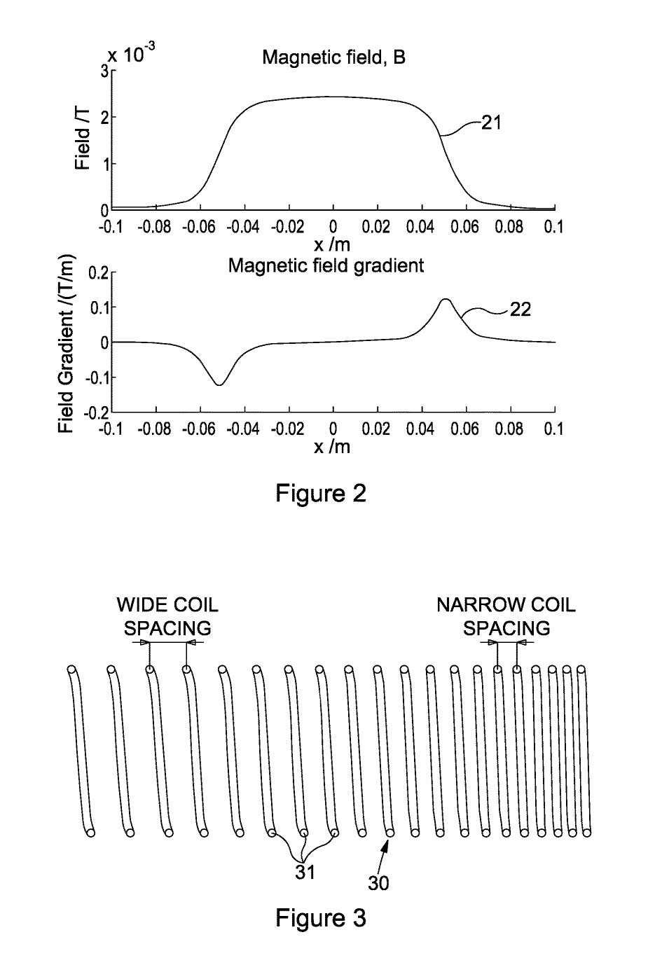

FIG. 2 is a graph of the magnetic field and field gradient along the axis of a conventional solenoid;

FIGS. 3, 4 and 5 show windings with varying pitch, varying number of layers, and varying radius, respectively;

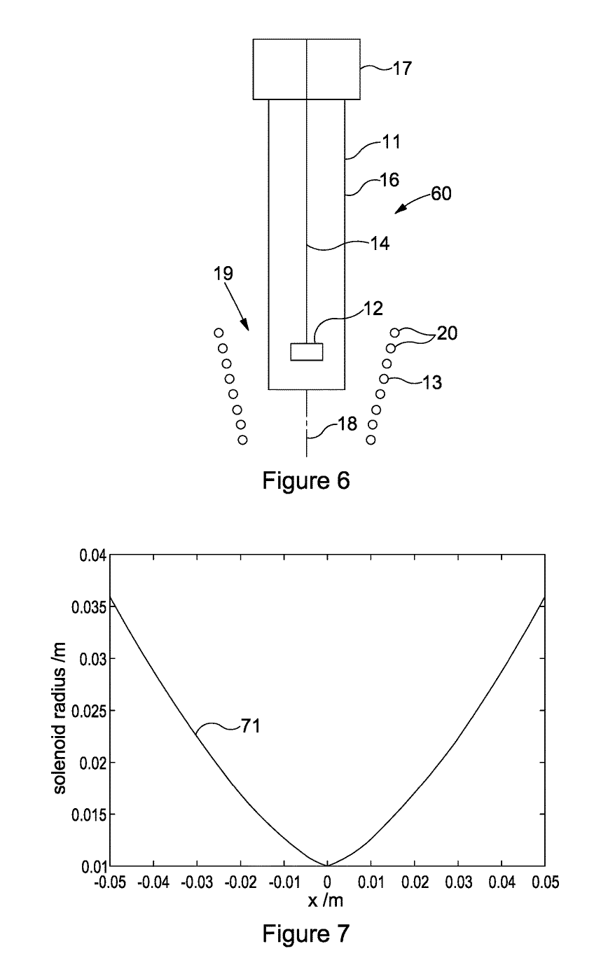

FIG. 6 is a schematic diagram of an apparatus according to an embodiment of the invention;

FIG. 7 is a graph of solenoid winding radius with respect to the distance from the solenoid centre along the solenoid axis, according to an embodiment,

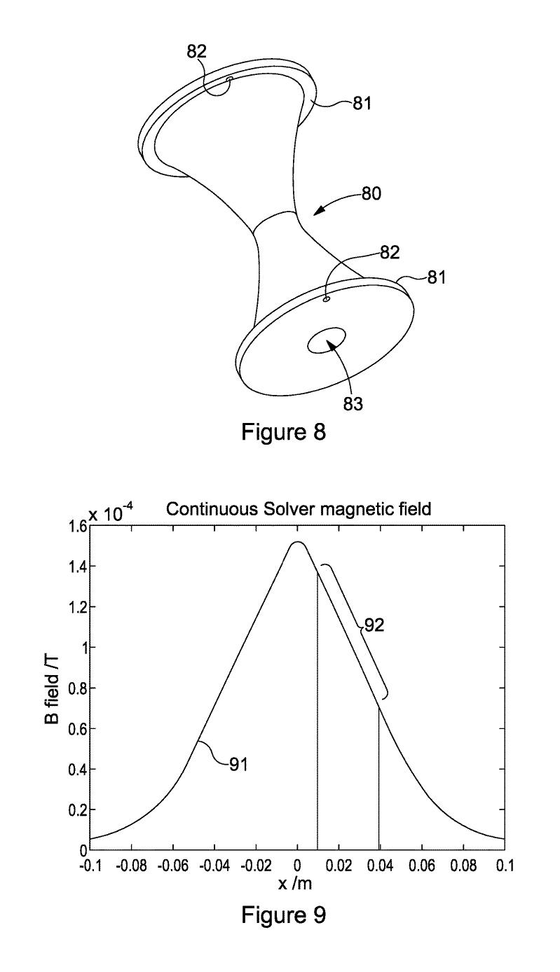

FIG. 8 is a schematic diagram of a solenoid former in accordance with FIG. 7;

FIG. 9 is graph of the modelled magnetic field with respect to the distance from the solenoid centre along the solenoid axis for the embodiment of FIGS. 7 and 8;

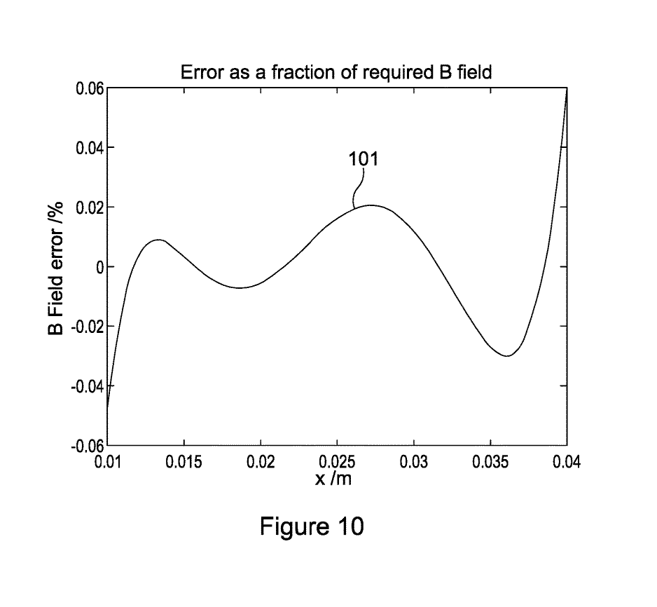

FIG. 10 is a graph of the variation from a uniform field gradient for a range of distances along the solenoid axis, from 1 cm to 4 cm from the solenoid centre; and

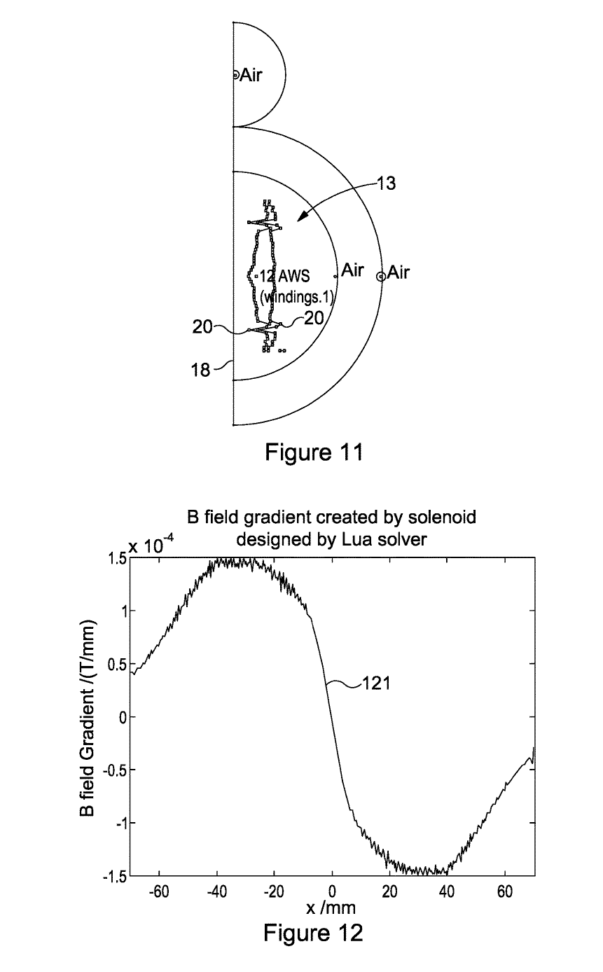

FIG. 11 is a schematic diagram of an alternative solenoid design, produced by optimisation via finite element modelling; and

FIG. 12 is a graph of the field gradient for the solenoid of FIG. 11.

Referring to FIG. 1, an arrangement 10 for mechanically loading a biological sample 1 is shown. The biological sample 1, in this case a tendon, is attached to the interior of a container 2 at a first location 11. At a second location 12, the sample 1 is attached to a magnet 3. The container 2 is then positioned over a solenoid 5 and a current passed through the solenoid 5 to control the force exerted by the magnet 3 on the sample 1.

In prior art arrangements a conventional solenoid 5 is used. The applicant has identified that a conventional solenoid 5 is highly sensitive to the position of the sample. It can be difficult to accurately position the sample within the container 2, because some of the sample 1 may be taken up by attaching it to the container 2 and magnet 3. Furthermore, the length of the sample 1 may change, for example as a result of creep from mechanical loading.

It is possible to compensate, at least to some extent, for the positional sensitivity of the actuation force by using closed loop control. Monitoring the force applied to the sample using load cell allows a current to be applied to the solenoid that produces a required force at the magnet. However, this approach requires a relatively expensive control loop and force transducer. Furthermore, it can only compensate for the positional sensitivity to a point—in some relative positions of magnet and solenoid, it may no longer be possible to exert the required force.

FIG. 2 shows a graph of modelled magnetic field 21 and magnetic field gradient 22 for an example conventional solenoid. The length of the solenoid is 100 mm, the winding radius is 10 mm, the winding has 51 turns, and the modelled current is 1 amp. It can be seen that the rate of change of magnetic field gradient with position is significant, and this is particularly pronounced where the field gradient is high (near the ends of the solenoid, at x˜±50 mm). This can be problematic in the case of mechanically loading a biological sample, because it results in inconsistency of loading. The present applicant has identified that it can be difficult to precisely position a ferromagnetic element (such as the magnet 3) when it is supported by a biological sample. It can be difficult to secure a biological sample 1 to the container 2 and magnet 3 such that the location of the magnet 3 within the container 2 is sufficiently accurate.

Furthermore, as the biological sample 1 undergoes mechanical loading, it may deform to a significant effect, for example as a result of creep. Even if the initial position of the magnet 3 is accurately defined within the container 2, as mechanical loading progresses over time, the location of the magnet 3 may move to a location in which the field gradient is substantially different than the initial position. The applicant has identified that it is this characteristic of the solenoid that results in the problematic inconsistency of loading of the biological sample.

According to an embodiment of the invention, the problem of inconsistency of mechanically loading a biological sample is solved by the use of a solenoid and ferromagnetic element in which the solenoid is configured to provide a more consistent force characteristic over a suitable distance.

This more consistent force characteristic can be approximated by configuring the solenoid to have a substantially uniform field gradient over a sufficient distance. For example, a solenoid with a field gradient that varies by less than 10% over a distance of at least 20 mm may provide a relatively uniform force (for a given current) on a ferroelectric element over that range of movement within the magnetic field of the solenoid. This configuration may be particularly applicable where the ferromagnetic element is relatively small.

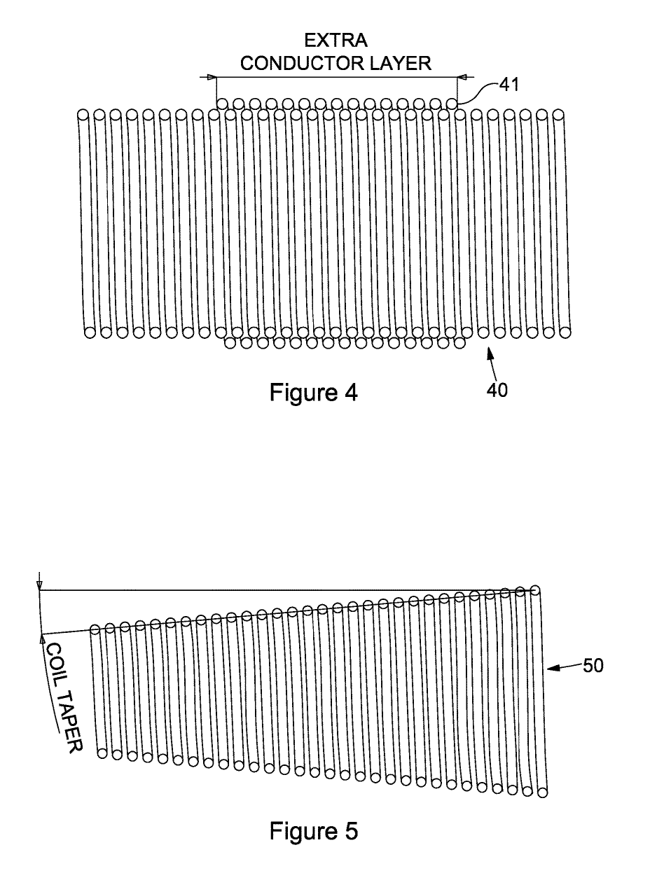

FIGS. 3 to 5 show examples of design parameters of a solenoid that affect the field of the solenoid. Increasing the pitch between adjacent turns 31 of a solenoid winding 30, as shown in FIG. 3, will decrease the magnetic field with increasing pitch. Adding additional layers of turns 41, as shown in FIG. 4, will increase the magnetic field in proportion to the number of layers. Adjusting the radius of the turns (or tapering the coil), as shown in FIG. 5, will increase the strength of the magnetic field at the solenoid axis with decreasing radius of the turns.

The relationship between such design parameters and the magnetic field and field gradient may be determined by methods such as finite element analysis and analytical approximations based on the Biot-Savart law.

Referring to FIG. 6, an apparatus 60 according to an embodiment is schematically illustrated, comprising a container 11, ferromagnetic element 12, and solenoid 13. A biological sample 14 is attached between the ferromagnetic element 12 and the container 11. In this example the biological sample is attached to the lid 17 of the container, but this is not essential, and the biological sample 14 may be supported in other configurations. For instance, the biological sample 14 could be supported by the container 11 at a first and second location, and the ferromagnetic element 12 supported by a portion of the biological sample 13 that is between the first and second location (e.g. in a lateral bridge configuration).

The container 11 comprises a body 16 and lid 17. The lid 17 is sealably engagable with the body 16, so that a sterile environment may be formed housing the biological sample 14. The lid 17 comprises gas exchange means 17a, in the form of small holes in the lid that permit gas exchange, allowing for adequate oxygenation of a cultured specimen in the container. The container 11 may be substantially cylindrical. The container 11 may comprise a test tube or centrifuge tube with a screw cap. The container 11 may be sterile.

The biological sample 14 may comprise tissue. The tissue may be cultured in vitro, or obtained by a biopsy or similar procedure from an organism. The biological sample 14 may comprise any biological sample. In one example the biological sample may be fascicles, such as tendon fascicles. The biological sample 14 may be surrounded by a growth or culture medium, for keeping the biological sample 14 viable.

The solenoid 13 in this example is configured to produce a field gradient that is more linear by varying the radius of the turns 20 of the winding with the location along the solenoid axis 18. The radius of the turns 20 increases towards the end of the solenoid 19 that is facing the container 11, so that the magnetic field on the axis of the solenoid 13 decreases towards the turns 20 with a larger radius.

The container 11 may be positioned completely outside the solenoid 13 (not shown), or positioned partly within the solenoid 13, as shown in FIG. 6. The solenoid may include a recess or through hole to facilitate this.

The ferromagnetic element 12 may comprise a permanent magnet, such as a rare earth magnet (e.g. cobalt samarium or neodymium). The ferromagnetic element 12 may be encapsulated with a biologically inert coating, which may be polymeric. An example of such a coating is polytetraflouroethylene (PTFE).

In this embodiment the field gradient of the solenoid 13 on the solenoid axis 18 is adjusted by varying the radius of the turns 20 of the solenoid. In other embodiments the pitch of the turns 20, or the number of layers in the solenoid 13 may be varied along the length of the solenoid 13 to achieve reduced variation in force as a function of the position of the ferromagnetic element 12. The turn pitch, number of layers and turn radii can each be adjusted independently to achieve the required coil configuration, or may be varied in any combination.

An illustrative example of a solenoid 13 that is suitable for providing a substantially uniform force on a ferromagnetic element over a particular distance (for a particular current flowing through the solenoid) is shown in FIGS. 7 and 8.

In this embodiment a uniform pitch of the turns is assumed, along with a uniform number of layers. The radius of the solenoid winding is varied so as to achieve a field gradient that varies by less than a predetermined amount along the axis of the solenoid over a predetermined distance. In this embodiment, the design of the solenoid was optimised to approximate a substantially uniform field gradient over a distance of between 10 mm and 40 mm from the centre of the solenoid.

Referring to FIG. 7, a curve 71 of the radius of the solenoid winding is shown with respect to the distance along the solenoid axis. In this example a minimum solenoid radius of 10 mm was selected, to allow the container to be positioned at least partly within the solenoid.

FIG. 8 shows a solid model of a solenoid former 80 in accordance with the graph of FIG. 7. Shoulders 81 are provided at the ends of the former 30, and through holes 82 are provided through these for securing the start and end of the solenoid winding. A through hole 83 is provided along the axis of the solenoid, for at least partially receiving the container.

FIG. 9 shows a curve 91 of calculated magnetic field on the solenoid axis for the solenoid design shown in FIGS. 7 and 8. It is clear that the graph is linear in the optimised region 92 between a distance of 10 mm and 40 mm from the centre of the solenoid. FIG. 10 shows a curve 101 of the difference between the magnetic field of the solenoid of FIGS. 7 and 8 and the linear target function against which the design was designed. An error of 0% would represent a (calculated) fixed field gradient for 10 mm<x<40 mm.

The design shown in FIGS. 7 and 8 is calculated to achieve a field gradient that varies by less than 0.1% over a predetermined distance of 30 mm (from x=10 mm to x=40 mm). It will be understood that a real solenoid made to this design may not produce a field that perfectly matches that calculated in FIGS. 9 and 10, but it can be expected that a relatively uniform force on a ferromagnetic element will result from the uniform field gradient over the predetermined distance. The ferromagnetic element may be arranged to extend over less than a fifth of the predetermined distance when the ferromagnetic element is supported by the biological sample. For instance, the ferromagnetic element for the embodiment of FIGS. 7 and 8 may have a length of 6 mm or less along the solenoid axis, when supported by a tissue sample.

Although a specific embodiment has been described in which a solenoid radius was varied to approximate a linear field gradient over a predetermined distance within the solenoid, in other examples the solenoid may be optimised with a particular ferromagnetic element in mind, so as to produce a particularly uniform force response with respect to the location of the ferromagnetic element along the solenoid axis.

Although a relatively small ferromagnetic element and a uniform field gradient is one way to achieve a more constant force characteristic over distance, an alternative approach is possible in which a ferromagnetic element extends over a greater distance along the solenoid axis. The appropriate configuration of the solenoid to approximate a constant force over a predetermined range of movement of the ferromagnetic element may thereby take into account the interaction of the ferromagnetic element with the magnetic field of the solenoid. Where the ferromagnetic element is a permanent magnet, the interaction between the distributed field of the magnet and that of the solenoid may be taken into account in optimising the solenoid to better approximate uniform force over a range of displacement.

Referring to FIG. 11, an alternative configuration for solenoid 13 is shown, in which the position of each turn of the windings 20 is illustrated with respect to the solenoid axis 18. This configuration was produced by iterative finite element modelling, using Lua scripting of FEMM (Finite Element Method Magnetics).

FIG. 12 shows a curve 121 of the modelled field gradient for the solenoid 13 of FIG. 11. The modelled field gradient is relatively uniform between x=20 mm and x=40 mm, varying by less than 15% over this range.

In an alternative arrangement, the solenoid may not be optimised to produce a linear field gradient. A relatively long ferromagnetic element may be used with a solenoid having a more typical construction (for example, having a substantially uniform radius, number of winding layers and turn pitch). The long ferromagnetic element may extend from a region through the end effect region of the solenoid, into a central region of the solenoid where the field is substantially uniform.

Using the example of FIG. 2, in a quiescent position the ferromagnetic element may extend from x=0 to x=150 mm. A movement of the ferromagnetic element of up to 40 mm along the solenoid axis could thereby be accommodated without moving the end within the coil out of the substantially uniform field region.

The magnetic force on the ferromagnetic element is proportional to the rate of change of the integral of the magnetic field over the region occupied by the ferromagnetic element. Arranging the ferromagnetic element to always include the end effect regions of the solenoid, while extending into the substantially uniform central field region may result in a linear increase in the field intersecting the element with movement along the axis—the increase coming from increasing the overlap in the uniform region. Such a configuration provides an alternative arrangement that approximates a uniform force over a range of movement, but may necessitate a longer container, to accommodate the ferromagnetic element.

The maximum force that can be produced on the ferromagnetic element by the solenoid is limited by the current that can flow through the solenoid without overheating it. This current limit can be increased by improving the cooling of the solenoid. This can be achieved in a number of ways, for instance: by using a material with high thermal conductivity for the bobbin or former; by providing structures to improve heat transfer to the ambient environment (e.g. fins and ridges); or by forced convection of a cooling fluid. The cooling fluid may be air or a liquid. which may comprise water. A fan or impeller may be provided to draw the fluid over the solenoid, thereby improving heat transfer from the solenoid winding. This may improve the maximum force that can be applied on the magnet using the solenoid.

The preceding examples are not intended to limit the scope of the invention. A number of variations are possible, within the scope of the invention, as determined by the appended claims.

1. An apparatus for mechanically loading a biological sample, comprising:
a container for housing the biological sample;
a ferromagnetic element, for attachment to the biological sample within the container; and
a solenoid for generating a magnetic field within the container, so as to apply a force to the ferromagnetic element, the solenoid having a solenoid axis;
wherein the solenoid is configured, when energised by a constant current, to produce a force on the ferromagnetic element that varies by less than 50% over a range of movement of the ferromagnetic element within the container of at least 10 mm.

2. The apparatus of claim 1, wherein the solenoid is configured to produce a field gradient that varies by less than 20% over a distance of at least 10 mm along the solenoid axis.

3. The apparatus of claim 1 or 2, wherein the solenoid comprises a winding with a radius that varies with position along the solenoid axis.

4. The apparatus of claim 3, wherein the radius of the winding increases with distance from the centre of the solenoid.

5. The apparatus of any preceding claim, wherein the solenoid comprises a winding that has a number of winding layers that varies with position along the solenoid axis.

6. The apparatus of any preceding claim, wherein the solenoid comprises a winding has a pitch that varies with position along the solenoid axis.

7. The apparatus of any preceding claim, further comprising means for cooling the solenoid by forced convection of a cooling fluid.

8. The apparatus of claim 7, wherein the fluid is air or water.

9. The apparatus of any preceding claim, wherein the container is a sterile container.

10. The apparatus of any preceding claim, wherein the container comprises a removable lid.

11. The apparatus of claim 10, wherein the lid comprises attachment means, for attachment to the biological sample.

12. The apparatus of any preceding claim, wherein the ferromagnetic element comprises a permanent magnet.

13. The apparatus of any preceding claim, wherein the ferromagnetic element comprises a biologically inert coating.

14. The apparatus of claim 13, wherein the coating is a polymeric material.

15. The apparatus of any preceding claim, wherein the solenoid has a hole or recess along the axis for at least partially receiving the container so that the ferromagnetic element within the container is within the solenoid.

16. The apparatus of claim 15, wherein the diameter of the hole or recess is at least 10 mm.

17. The apparatus of claim 15 or 16, wherein the depth of the hole or recess is at least 20 mm.

18. The apparatus of any preceding claim, wherein the length of the solenoid is at least 50 mm.

19. The apparatus of any preceding claim, wherein the container comprises gas exchange means.

20. A method of mechanically loading a biological sample, comprising:
attaching a first portion of a the biological sample to a fixed object;
attaching a ferromagnetic element to a second portion of the biological sample;
positioning the ferromagnetic element within a working region of a solenoid; and
exerting a force on the ferromagnetic element using a solenoid;
wherein the solenoid is configured, when energised by a constant current, to produce a force on the ferromagnetic element that varies by less than 50% over a range of movement of the ferromagnetic element within the container of at least 10 mm.

21. The method of claim 20, wherein the method comprises using the apparatus according to any preceding claim.

22. The method of any preceding method claim, including a step of testing a substance for use a medicament on the biological sample while it is loaded periodically using the solenoid.

23. The method of any preceding method claim, wherein the biological sample is tissue, and the method comprises conditioning the tissue using the force exerted by the solenoid.

24. An apparatus substantially as hereinbefore described, with reference to accompanying FIGS. 3 to 12.

25. A method substantially as hereinbefore described, with reference to the accompanying FIGS. 3 to 12.
